(19) United States
(12) Patent Application Publication
(10) Pub. No.: US 2003/0055438 A1
(43) Pub. Date: Mar. 20, 2003

(54) LIGATING INSTRUMENT

(75) Inventor: Minoru Hirata, Ichikawa-city (JP)

Correspondence Address: JORDAN AND HAMBURG LLP, 122 EAST 42ND STREET, SUITE 4000, NEW YORK, NY, 10168, US

(73) Assignee: Hirata Precisions Co., Ltd., Ichikawa-city (JP)

(21) Appl. No.: 10/059,861

(22) Filed: Jan. 29, 2002

(30) Foreign Application Priority Data
Sep. 18, 2001 (JP) 2001-282819

(51) Int. Cl. A61B 17/12

(52) U.S. Cl. 606/139; 606/144

(57) ABSTRACT

By operating a lever 29, a slide rod 11 and an inter locking rod 12 operatively associated therewith in an actuator body 2 are moved, by which a bobbin 21 is fitted in a movable member 9 projecting out of a guide tube 6 and connected to the slide rod 11. And a ligature 60 is wound on a spool formed in the movable member 9. The movable member 9 is moved backward to disengage therefrom the bobbin 21 to form a loop of the ligature 60 to form a knot.

### BACKGROUND OF THE INVENTION

1. Field of the Invention

The present invention relates to a simple-structured ligating instrument for use in endoscopic surgical procedures.

2. Description of the Prior Art

In this specification all the acts of tying, and the like will be generically called “ligation” and ligation with two or more knots will be called “multiple-knots ligation.” The multiple-knots ligation is roughly classified into sailor's knot, granny knot and surgical knot. The surgical knot is shown in FIG. 1, in which a ligature is wound around it twice to form a first knot A (double) to frictionally prevent the ligature from slackening off and then the ligature is wound once in a direction opposite to that for the first knot A to form a second knot B (single). This ligation keeps the ligature taut, and hence it is applied to an operative site in the body where the ligature is strained. The sailor's knot is shown in FIG. 2, in which first and second knots A and B are both single but different in direction. This is simpler than any methods of other ligations and does not easily become loose; hence, this ligation is customarily used to stop the bleeding, for instance. In the granny knot, the first and second knots A and B are both single like in the case of the sailor's knot but both of them is made in the same direction. FIG. 3 shows a knots ligation, in which first and second knots A and B are formed by the method of sailor's knot ligation, and a third knot ligation C is added with a view to preventing slackening of the ligature. Incidentally, quadruple-, quintuple-, and further multi-knots ligations will provide increased reliability irrespective of the kinds of ligatures used.

A conventional ligating instrument for use in endoscopic operative procedures is disclosed, for example, in Japanese Patent Application Publication No. 201767/98. The prior art instrument is disadvantageous in that the winding of a ligature on a spool is so much complicated and difficult and that the ligature wound on the spool easily becomes unwound. As a solution to such problems, the inventor has previously proposed a novel ligating instrument in Japanese Patent Application No. 370007/99. However, the inventor's proposed instrument also had the disadvantage of a complicated-structured bobbin because of the provision of a ligature insertion groove in its guide frame portion. Further, the bobbin to be fitted in a movable member has at its tip end portion a provisional ligature retaining part and at its base end portion a means for detachably fitting the bobbin into a guide tube, and this fitting means has a construction by which a hanging hook for insertion mounted on a bobbin connecting portion is engaged with a support shaft member in the guide tube to hold therein the bobbin—this also increased complexity of the bobbin structure. With such a construction, fitting the bobbin into the above-said tube is troublesome and time-consuming. Accordingly, the inventor's proposed ligating instrument is not suitable for ligation at the time of surgery that must be done quickly in an urgent situation.

### SUMMARY OF THE INVENTION

It is therefore an object of the present invention to provide a simple-structured ligating instrument that permits easy and quick attachment and detachment of the bobbin.

Another object of the present invention is to provide a ligating instrument that allows simplification of the bobbin to facilitate disengagement therefrom of a ligature by movement of a movable member.

Still another object of the present invention is to provide a ligating instrument that permits smooth operation of a pawl associated with a inter locking rod for the insertion of the bobbin into the movable member to ensure removal of the bobbin from the movable member during ligation.

To attain the above objective, the ligating instrument of according to an aspect of the present invention comprises:

A acutuator body composed of a actuator frame with actuating members disposed therein and a hand grip extending integrally from the underside of the base end portion of the actuator frame;

a guide tube inserted at one end portion in the actuator body and projecting out at the other end portion from the forward end of the actuator body;

a movable member inserted into the guide tube, connected at one end to a slide rod and projecting out at the other end portion from the forward end of the guide tube, the movable member having in its tip end portion a ligature retaining portion;

a slide rod connected to the movable member for horizontal movement as a unitary structure therewith;

an inter locking rod disposed in parallel to the slide rod and having formed in its top an engaging serrated part;

a pawl associated with the slide rod for engagement with the engaging serrated part thereof,

a bobbin fitted into the movable member, fixed in the guide tube with lugs on both sides of its base end portion engaged with slits formed in the guide tube and having a plurality of equally spaced guide frames provited on the base board, the bobbin being inserted into the movable member to form a spool in the movable member and guiding a ligature wound on the spool to come it off when the bar is moved;

a lever projecting downwardly from the actuator body; and

a vertically moving member adapted to move up and down through a cam mechanism coupled to a rotary member pivoted to the lever when the lever is operated;

wherein, by the upward movement of the vertically moving member, the pawl is brought into engagement with a one-step-forward serration of the engaging serrated part to thereby slide back the movable member by the same pitch as the spacing of the guide frame of said bobbin.

According to another aspect of the invention, the movable member is a thin, flat member and has in its forward end portion, as the ligature retaining portion, a circular through hole with a notch for hooking the ligature.

According to another aspect of the invention: the pawl has a lug disposed at its upper portion and supported through a spring to a mounting member composed of a tubular member with the inter locking rod inserted therethrough and a forked mounting part disposed on said tubular member; the pawl is pivoted at its base end portion to the based end portion of the mounting part; the mounting part has in its back a groove aslant toward the forward end portion of the movable member so that the vertically moving member having a lug on its top end portion for abutment wit the lug of the pawl moves up and down along the groove.

According to another aspect of the invention, a reciprocating motion support member of an inverted L-shaped cross-section is provided at right angles to the vertically moving member and has a slide surface that makes sliding contact with a step pin of the pawl mounting member.

According to another aspect of the invention, the inter locking rod has in its underside a positioning serrated part corresponding to the engaging serrated part, for causing the pawl to slide on the inter locking rod on a step-by-step basis to ensure smooth and accurate positioning of the inter locking rod during its reciprocating motion by the sliding motion of the movable member.

According to still another aspect of the invention, a forwardly-upward-directed button is attached to the top of a spring-loaded shaft connected to the base end portion of the slide rod and adapted to push out the bobbin from the movable member when the button is pulled.

### BRIEF DESCRIPTION TF DRAWINGS

FIG. 1 is a diagram schematically showing a surgical ligation;

FIG. 2 is a diagram schematically showing a sailor's ligation;

FIG. 3 is a diagram schematically showing a triple-knot ligation;

FIG. 4 is a perspective view of the ligating instrument according to the present invention;

FIG. 5 is its side view;

FIG. 6 is its longitudinal-sectional view;

FIG. 7 is a perspective view of a bobbin for use in the present invention;

FIG. 8 is a front view depicting the bobbin, a movable member and a guide tube separately;

FIG. 9 is a side view showing the state in which the movable member is inserted in the guide tube;

FIG. 10 is a side view showing the state in which the bobbin is inserted in the guide tube;

FIG. 11 is a side view showing the state in which the bobbin is held in the guide tube;

FIG. 12 is a diagram explanatory of the sliding movement of a pawl and a vertically moving member relative to each other;

FIG. 13 is a side view showing the state in which a ligature is tied up in the bobbin;

FIG. 14 is a side view showing the state in which a ligature connected to a needle is attached to a retaining portion at the tip end portion of the movable member;

FIG. 15 is a diagram showing a step involved in surgical ligation;

FIG. 16 is a diagram showing another step in surgical ligation;

FIG. 17 is a diagram showing another step in surgical ligation;

FIG. 18 is a diagram showing still another step in surgical ligation;

FIG. 19 is a diagram showing a step involved in sailor's ligation;

FIG. 20 is a diagram showing a step involved in continuous stitching;

FIG. 21 is a diagram showing another step in continuous stitching;

FIG. 22 is a diagram showing another step in continuous stitching; and

FIG. 23 is a diagram showing still another step in continuous stitching.

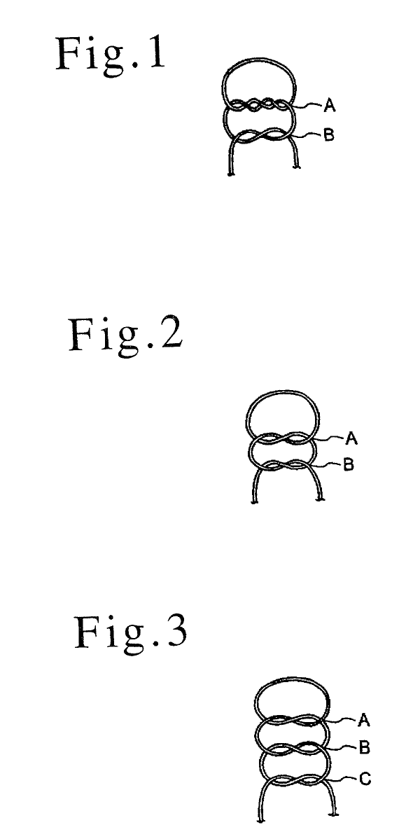

### DETAILED DESCRIPTION OF THE PFREFERRED EMBODIMENT

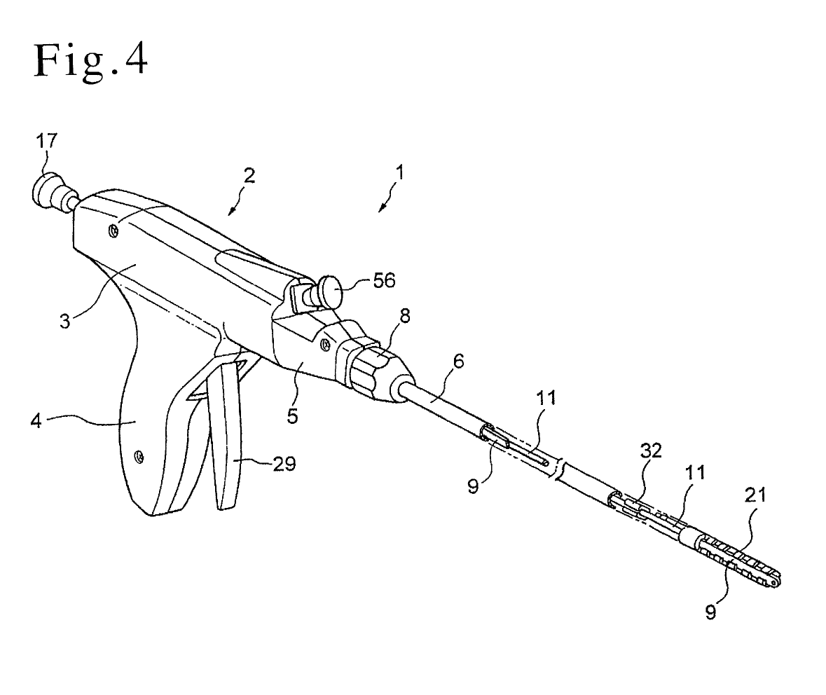

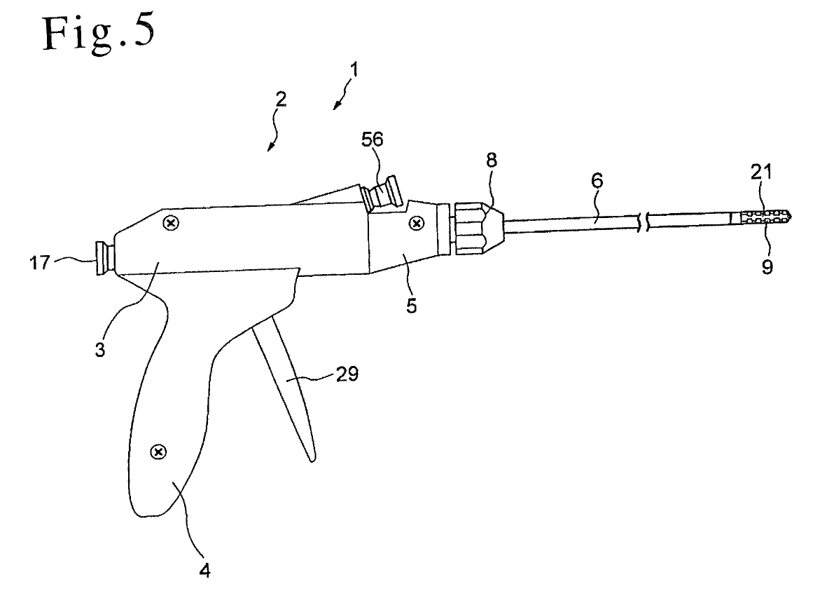

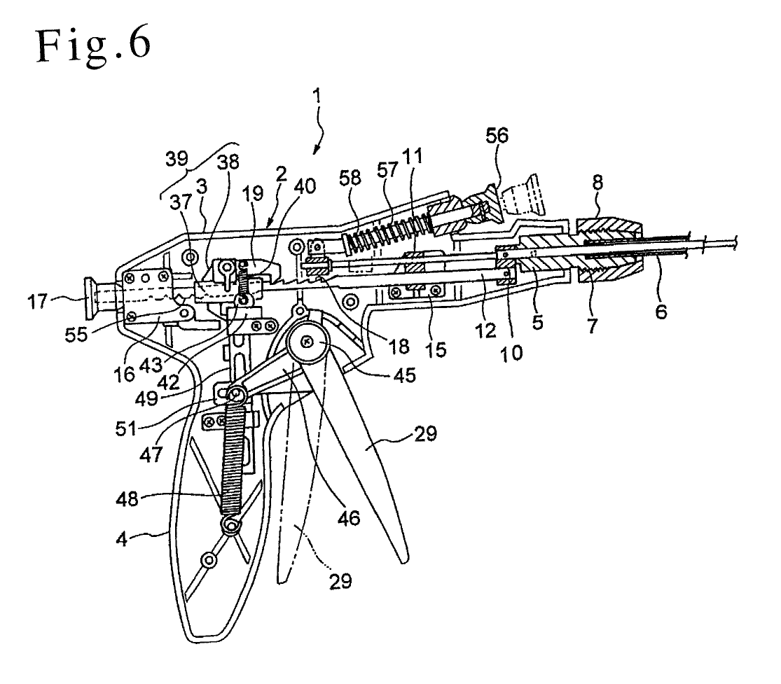

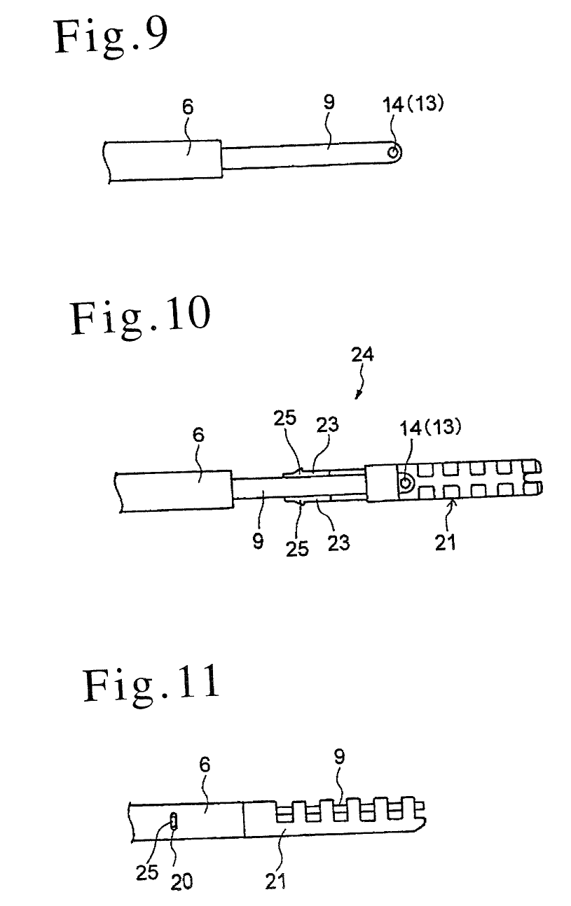

FIG. 4 is a perspective view illustrating the entire structure of the ligating instrument according to an embodiment of the present invention, FIG. 5 is its side view and FIG. 6 is its longitudinal-sectional view. An actuator body 2 of the ligating instrument, indicated generally by 1, is a pistol-shaped screw-held structure or assembly of longitudinally divided shell halves as shown. The actuator body 2 is composed of an actuator frame 3 having disposed therein actuator members described later on and a hand grip 4 extending down from the actuator frame 3. Reference numeral 5 denotes a guide tube fixing means projecting out from the forward end of the actuator frame 3. The fixing means 5 has a protrusion (not shown) for engagement with an elongated notch (not shown) made in the rearward end portion of a guide tube 6 to fixedly hold it in position. Reference numeral 7 denotes an externally threaded shaft, which extends integrally from the fixing means 5. The guide tube 6 is secured to the forward end of the actuator frame 3 by tightening a nut 8 on the threaded shaft 7. Reference numeral 9 denotes a thin, flat movable member inserted into the guide tube 6 through the fixing means 5. The movable member 9 has its base end portion connected by a forked connecting member 10 to a slide rod 11 similarly inserted in the guide tube 6. Reference number 12 denotes a inter locking rod extending in parallel to the slide rod 11 and having an engaging serrated part 18 in its top surface for engagement with a spring-biased pawl 19 described later on. The movable member 9 has at its end portion a ligature retaining portion 13, which is shown to have a circular through hole 14 as depicted in FIGS. 8 and 9. The ligature is tied up through the hole 14. The through hole 14 may be notched to hook the ligature. Reference numeral 18 denotes an engaging serrated part in it's top surface for engagement with a spring-biased pawl 19. In this embodiment, the engaging serrated part 18 have plural number (in this embodiment, the number of the engaging serrated part 18 is five). The inter locking rod connecting member 10 has its forward end position connected to the lower part of the connecting member 10 and extends rearward through a bearing 15 and then through a sleeve 37 and has its rear end secured to a push button 17 externally of the actuator body 2. With the button 17 pushed, the inter locking rod 12 is moved forward together with the slide rod 11 and the movable member 9.

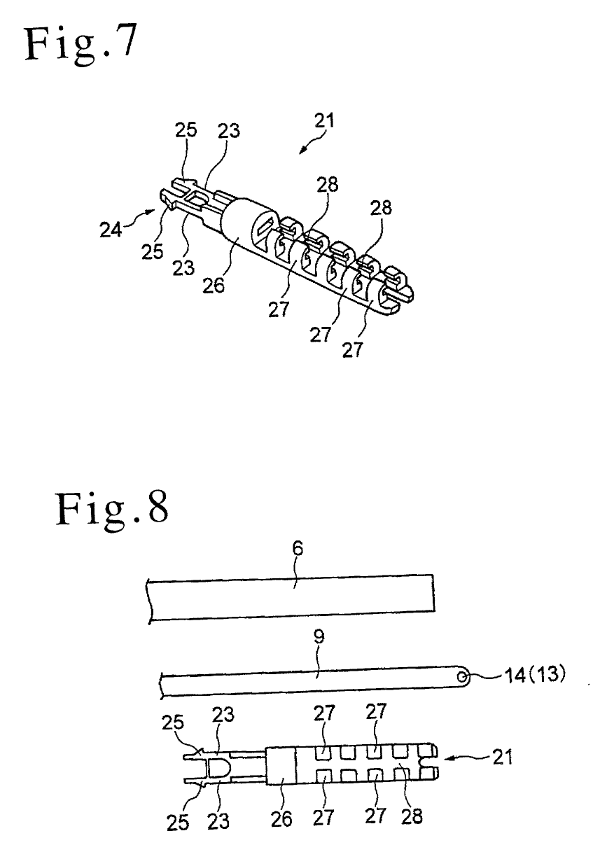

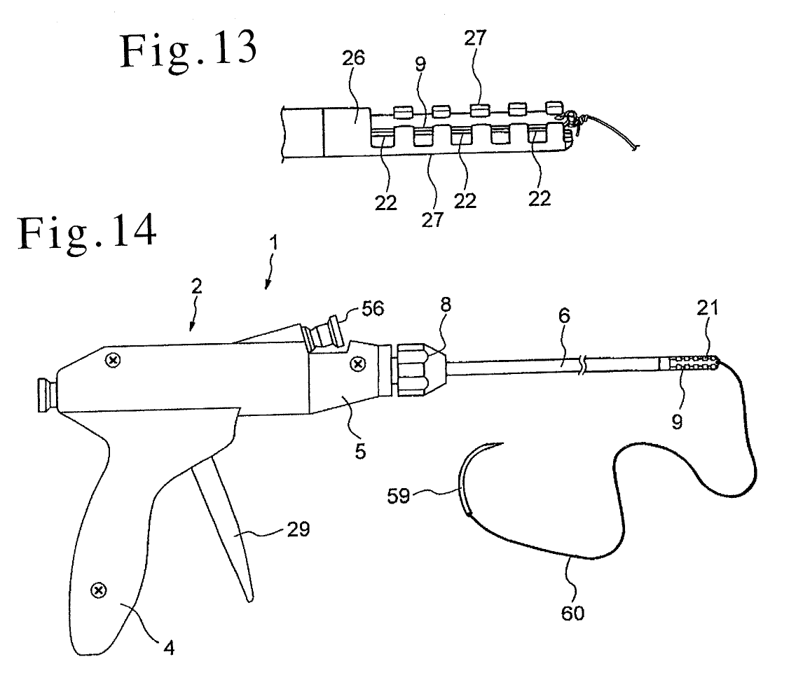

Turning next to FIG. 7, reference numeral 21 denotes a bobbin engaged with the tip of the movable member 9 and having its tip end portion inserted and fixed in the guide tube 6. When the movable member 9 is moved back by rearward movement of the slide rod 11, the bobbin 21 guides the movable member 9 so that a ligature wound on each spool 22 (FIG. 13) of the movable member 9 is removed therefrom as described later on. As shown in FIG. 7, the bobbin 21 has at its rear end a forked engaging portion 24 with shafts 23 and protrusions protrusively provided on outer side surfaces of the shafts 23 for engagement with counterparts of the guide tube 6 when the bobbin 21 is inserted in the guide tube 6. Reference numeral 26 denotes a dome-shaped support frame contiguous to the engaging portion 24 to ensure coupling of the bobbin 21 with the movable member 9. Reference numeral 27 denotes guide frames integral with the bobbin body. The guide frames 27 each have substantially an elliptic cross-section and a cutout 28 in the top. In this embodiment, the number of guide frames 27 is five. When the movable member 9 is inserted into the bobbin 21 the cutouts 28 cooperate with the guide frames 27 to form the spools of the movable member 9 as described later on; when the movable member 29 is moved back, the ligature wound on the spool 22 is guided off as mentioned above.

FIG. 8 shows the bobbin 21, the movable member 9 and the guide tube 6 separately; FIG. 9 is a side view showing the state in which the movable member 9 is inserted in the guide tube 6; FIG. 10 is a side view showing the state in which the bobbin 21 receives the movable member 9 inserted therein; and FIG. 11 is a side view showing the state in which the bobbin 21 is fitted in the guide tube 6. That is, the movable member 9 is urged to project out forwardly of the guide tube 6 by pulling a hand lever 29 and pressing the push button 17. And the engaging portion 24 of the bobbin 21 with its support frame 26 and guide frames 27 receiving the movable member 9 is inserted into the guide tube 6. As a result, the engaging protrusions 25 of the bobbin 21 slide into slits 20 cut in the guide tube 6, fixedly supporting thereto the bobbin 21 together with the movable member 9 as depicted in FIG. 11. Such a structure permits simple, easy and quick attachment of the bobbin 21 to the guide tube 6 without the need for using a provisional ligature retainer and so forth unlike in the prior art. In FIG. 4, reference numeral 32 denotes an ejector head secured to the slide rod 11, and 56 denotes button. Pulling the button 56, the ejector head 32 is moved forward to disengage the bobbin 21 from the guide tube 6 as depicted in FIG. 4. In this instance, the bobbin 21 and the ejector head 32 attached to the slide rod 11 have the same length so that the bobbin 21 normally projects out forwardly of the tip end portion of the guide tube 6.

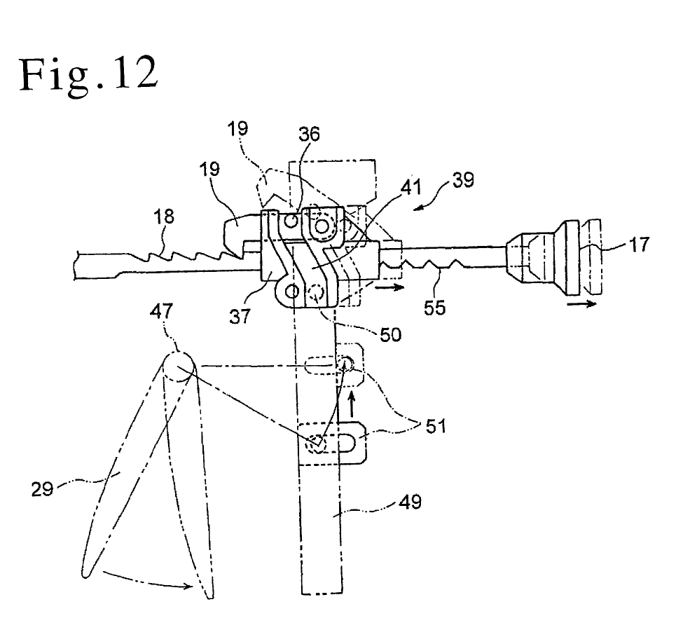

Next, a description will be given of the mechanism by which the movable member 9 is slid along the bobbin 21 at the same pitch as the spacing of the guide frames 27. Reference numeral 19 denotes the afore-mentioned pawl that engages the engaging serrated part 18 of the inter locking rod 12. The pawl 19 has a lug 36 protrusively provided on one side thereof near the upper marginal edge and is elastically supported by a spring 40 to a mounting member 39 consisting of a tubular member 37 through which the inter locking rod 12 passes and a forked mounting part 38 disposed on the tubular member 37. That is, the inter locking rod 12 is inserted through the tubular member 37 in a manner to be movable back and force. The pawl 19 is pivoted to the base end portion of the mounting part 38, and the back of the mounting part 38 has a groove 41 a little aslant toward the forward end portion of the movable member 9 (see FIG. 12). Reference numeral 42 denotes a reciprocation support member 42 of an inverted L-shaped cross-section, which is disposed at right angles to a vertically moving member 49 and has a sliding surface that makes sliding contact with the mounting member 39 along a stop pin 43. The reciprocation support member 42 permits smooth and accurate reciprocating motion of the pawl 19 along the support member 42 in parallel thereto. That is, the lever 29 is pivotally mounted on the underside of the actuator frame 3 substantially centrally thereof as indicated by the pivot 45. Extended from the pivot 45 of the lever 29 is a rotary member 46. A spring 48 is located between the extreme end of the rotary member 46 and a pin 47 provided near the upper end of the hand grip 4. The above-mentioned vertically moving member 49 which is disposed in the hand grip 4 is pivoted to the pin 47 of the rotary member 46 and has a lug 50 above it and come in contact against the underside of the pawl mounting member 39. A horizontal cam 51 with an elongated hole is mounted on the vertically moving member 49 substantially centrally thereof at right angles thereto. A pin provided at the tip end portion of the rotary member 46 engages in the elongated hole of the horizontal cam 51. As the lever 29 is operated, the vertically moving member 49 moves up through the horizontal cam 51, by which the pawl 19 is pushed up and out of engagement with a first serration of the serrated part 18 of the inter locking rod 12, and then the pawl 19 engages therewith again but with a second serration forward of the fist one by the return elasticity of the springs 40, 48 thereby pulling back the inter locking rod 12 accordingly. As a result, the movable member 9 operatively associated with the inter locking rod 12 also moves back toward the rear end portion of the actuator body 2 by the spacing of the guide frames 27 of the bobbin 21.

Reference numeral 55 denotes a positioning serrated part provided in the underside of the inter locking rod 12, which corresponds to the engaging serrated part 18. The positioning serrated part 55 causes the pawl 19 to slide on the inter locking rod 12 on a stepwise basis, ensuring smooth and accurate positioning of the inter locking rod 12 during its reciprocating motion by the sliding motion of the movable member 9.

In FIGS. 4 to 6, reference numeral 56 denotes the afore-mentioned button attached to the top of a spring-loaded shaft 57 connected to the base end portion of the slide rod 11. The button 56 is disposed forwardly upward to the acutuator frame 3. On pulling the button 56 against the tensile force of a spring 58 wound around the shaft 57, the slide rod 11 moves forward, and the ejector rod 32 pushes out of the guide tube 6 the bobbin 21 stained with blood or the like for replacement with a new bobbin 21.

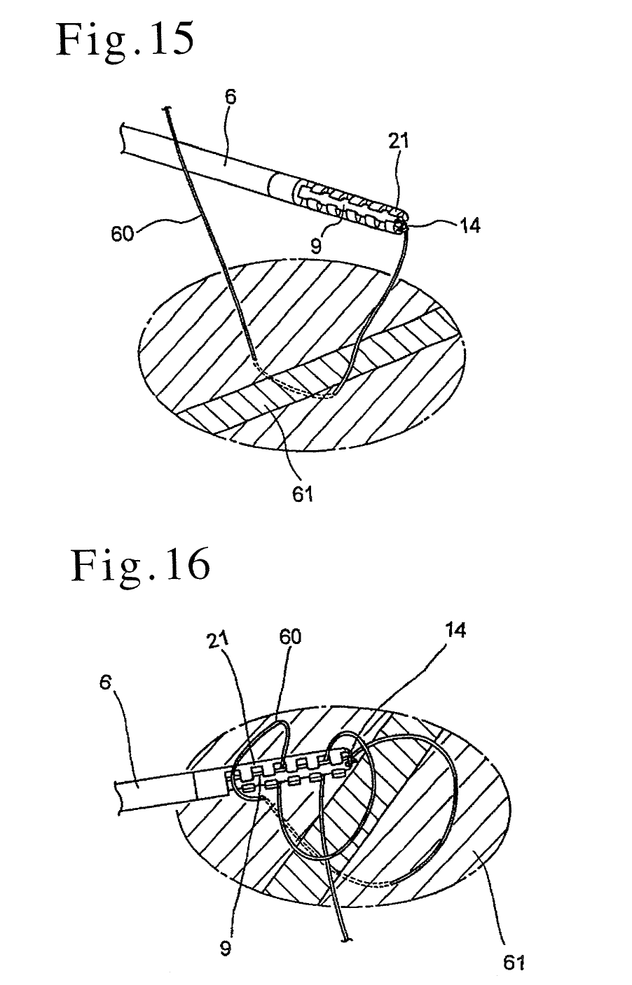

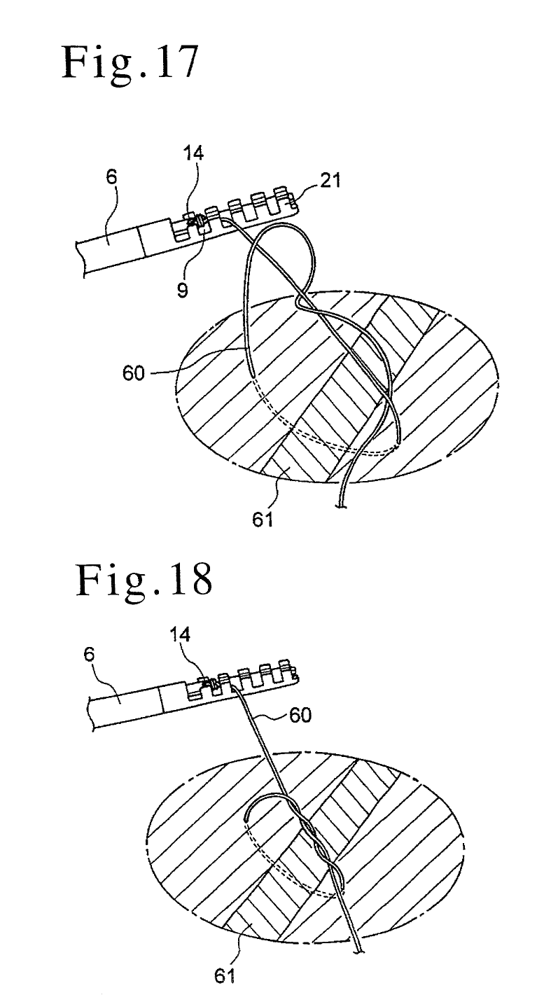

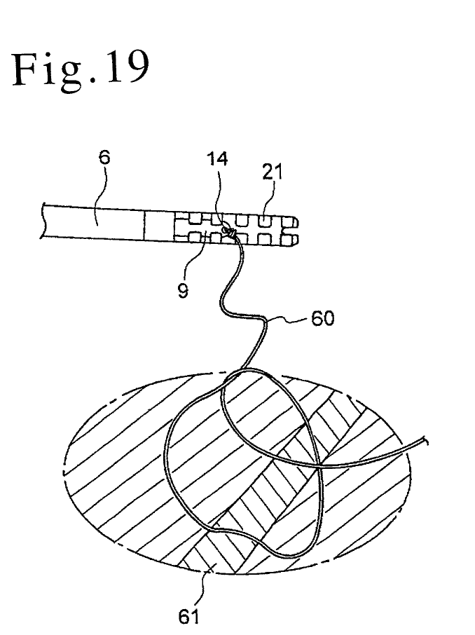

Next, a description will be given of how to use the ligating instrument in endoscopic surgery or the like. In the first place, a ligature 60 connected to a needle 59 is passed through the ligature retaining portion at the tip of the movable member 9 to tie a knot (see FIGS. 13 and 14). Then, body tissue 61 is stitched once with the ligature 60 from the right-hand to the left-hand direction with respect to the tissue 61 as depicted in FIG. 15. After this, the ligature 60 with the needle 59 is pulled out of the bobbin 21 toward an operator. Then, the bobbin 21 is inserted in the movable member 9 and the ligature 60 is passed once through the gap of the guide frame 27 third from the forward end of the bobbin 21 from above and then once through the gap of the first guide frame 27 of the bobbin 21 from above to thereby form a double loop. In this case, when the needle 59 can easily be passed through the gaps by passing the rear end of the needle 59 first through the gaps, not needle tip. Next, upon operating the lever 29 three times in succession, the movable member 9 moves back three pitches and disengages from the bobbin 21. By pulling the ligature 60 clockwise in this state, a surgical knot is tied (first knot) (see FIG. 18).

A sailor's ligation method will be described. In this instance, the ligature 60 connected to the needle 59 is passed from above through unused gap between the movable member 60 and the bobbin 21. Then, the movable member 9 is moved one or two pitches to a fourth or fifth guide frame 27 of the bobbin 21. Then, pulling counterclockwise the ligature having come off the bobbin 21, the bilateral knot is formed (second knot).

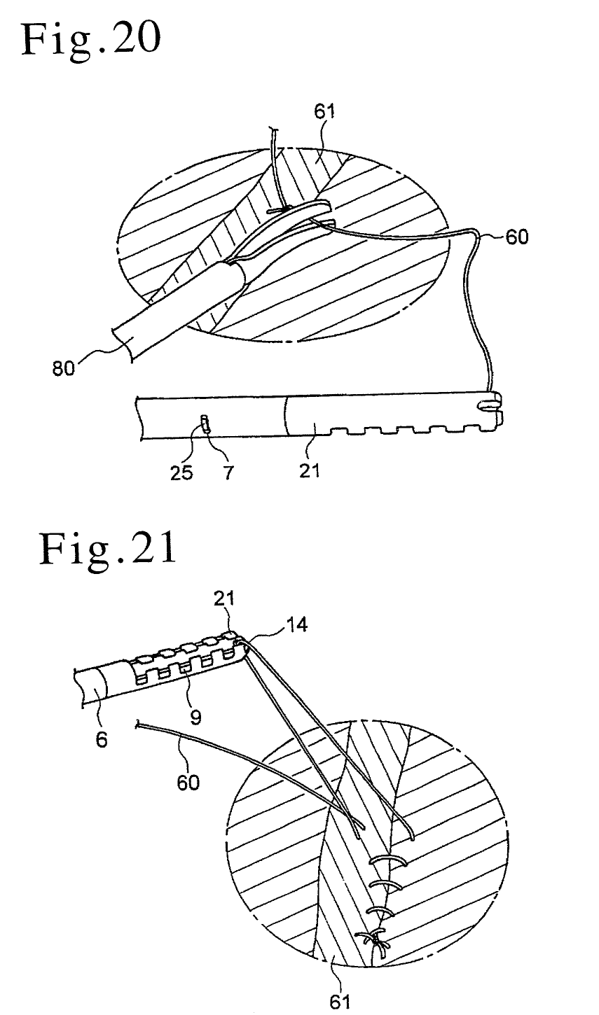

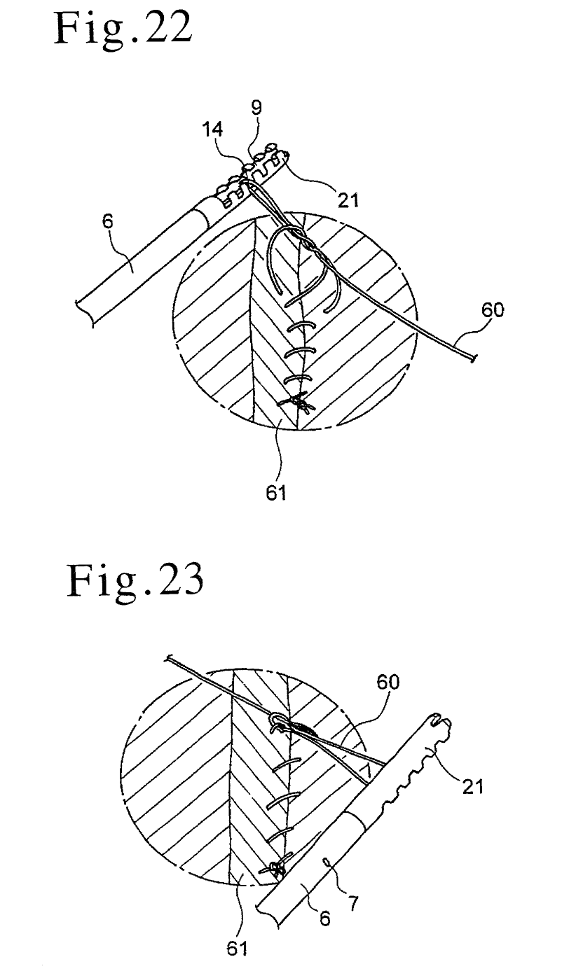

The sailor's ligation can be followed by continuous stitching after cutting the ligation connected to the movable member 9 with a cutter 80 (see FIG. 20). After final stitching, press the push button 17 while holding the lever 29 in the operated position, the movable member 9 is moved to the forward end of the guide tube 6. Then, after passing the ligature 61 through the retaining portion 13 at the tip end portion of the movable member 9, body tissue is stitched once (FIGS. 20 and 21). At this time, after the stitching, the ligature 60 is taken out of the body and a knot is tied, then the knot is returned by an instrument into the operative site to ligate and is tightened; after the subsequent stitching, the ligation can be achieved at the operative site by the ligating instrument of the present invention. After this, ligation is carried out following the surgical and sailor's ligation procedures (see FIGS. 22 and 23).

### EFFECT OF THE INVENTION

With the construction described above, the present invention offers simple-structured ligating instrument that permits easy and rapid attachment and detachment of the bobbin to and from the instrument body. Further, since the invention simplifies the configuration of the bobbin, the ligating instrument allows simple and easy unhooking of the ligature by the movement of the movable member and facilitates the operation of the pawl associated with the inter locking rod and hence ensures removal of the bobbin from the movable member during ligation. Moreover, the provision of the reciprocating-motion support member for smooth movement of the pawl ensures accurate movement of the movable member.

What is claimed is:

1. A ligating instrument comprising:
an acutuator body composed of a actuator frame with actuating members disposed therein and a hand grip extending integrally from the underside of the base end portion of the actuator frame;
a guide tube inserted at one end portion in the actuator body and projecting out at the other end portion from the forward end of the actuator body;
a movable member inserted into the guide tube, connected at one end to a slide rod and projecting out at the other end portion from the forward end of the tube, the movable member having in its tip end portion a ligature retaining portion;
a slide rod connected to the movable member for horizontal movement as a unitary structure therewith;
an inter locking rod disposed in parallel to the slide rod and having formed in its top an engaging serrated part;
a pawl associated with the slide rod for engagement with the engaging serrated part thereof;
a bobbin fitted into the movable member, fixed in the guide tube with lugs on both sides of its base end portion engaged with slits formed in the guide tube and having a plurality of equally spaced guide frames provited on the base board, the bobbin being inserted into the movable member to form a spool in the movable member and guiding a ligature wound on the spool to come it off when the inter locking rod is moved;
a lever projecting downwardly from the actuator body; and
a vertically moving member adapted to move up and down through a cam mechanism coupled to a rotary member pivoted to the lever when the an actuator body composed of an actuator frame with actuating members disposed therein and a hand grip extending integrally from the underside of the base end portion of said actuator frame;
a guide tube inserted at one end portion in said actuator body and projecting out at the other end portion from the forward end of said actuator body;
a movable member inserted into said guide tube, connected at one end to a slide rod and projecting out at the other end portion from the forward end of said guide tube, said movable member having in its tip end portion a ligature retaining portion;
a slide rod connected to said movable member for horizontal movement as a unitary structure therewith;
a inter locking rod disposed in parallel to said slide rod and having formed in its top an engaging serrated part;
a pawl associated with said slide rod for engagement with said engaging serrated part thereof;
a bobbin fitted into said movable member, fixed in said guide tube with lugs on both sides of its base end portion engaged with slits formed in said guide tube and having a plurality of equally spaced guide frames, said bobbin being inserted into said movable member to form a spool in said movable member and guiding a ligature wound on said spool to come it off when said inter locking rod is moved;
a lever projecting downwardly from said actuator body; and
a vertically moving member adapted to move up and down through a cam mechanism coupled to a rotary member pivoted to said lever when said lever is operated;
wherein, by the upward movement of said vertically moving member, said pawl is brought into engagement with a one-step-forward serration of said engaging serrated part to thereby slide back said movable member by the same pitch as the spacing of said guide frame of said bobbin.

2. The instrument of claim 1, wherein said movable member is a thin, flat member and has in its forward end portion, as said ligature retaining portion, a circular through hole with a notch for hooking said ligature.

3. The instrument of claim 1 or 2, wherein: said pawl has a lug disposed at its upper portion and supported through a spring to a mounting member composed of a tubular member with said bar inserted therethrough and a forked mounting part disposed on said tubular member; said pawl is pivoted at its base end portion to the based end portion of said mounting part; said mounting part has in its back a groove aslant toward the forward end portion of said movable member so that said vertically moving member having a lug on its top end portion for abutment wit said lug of said pawl moves up and down along said groove.

4. The instrument of claim 1 or 2, further comprising a reciprocating motion support member of an inverted L-shaped cross-section provided at right angles to said vertically moving member and having a slide surface that makes sliding contact with a step pin of said pawl mounting member.

5. The instrument of any one of claims 1 to 3, wherein said inter locking rod has in its underside a positioning serrated part corresponding to said engaging serrated part, for causing said pawl to slide on said inter locking rod on a step-by-step basis to ensure smooth and accurate positioning of said inter lockng rod during its reciprocating motion by the sliding motion of said movable member.

6. The instrument of any one o claims 1 to 5, further comprising a forwardly-upward-directed button attached to the top of a spring-loaded shaft connected to the base end portion of said slide rod and adapted to push out said bobbin from said movable member when said button is pulled.
